(19) United States
(12) Patent Application Publication
(10) Pub. No.: US 2021/0386346 A1
(43) Pub. Date: Dec. 16, 2021

(54) MAGNETOENCEPHALOGRAPH AND BRAIN'S MAGNETIC FIELD MEASUREMENT METHOD

(71) Applicants: HAMAMATSU PHOTONICS K.K., Hamamatsu-shi (JP); Kyoto University, Kyoto-shi (JP)

(72) Inventors: Takahiro MORIYA, Hamamatsu-shi (JP); Takenori OIDA, Hamamatsu-shi (JP); Akinori SAITO, Hamamatsu-shi (JP); Motohiro SUYAMA, Hamamatsu-shi (JP); Tetsuo KOBAYASHI, Kyoto-shi (JP)

(73) Assignees: HAMAMATSU PHOTONICS K.K., Hamamatsu-shi (JP); Kyoto University, Kyoto-shi (JP)

(21) Appl. No.: 17/346,331

(22) Filed: Jun. 14, 2021

(30) Foreign Application Priority Data
Jun. 16, 2020 (JP) 2020-103960

(51) Int. Cl. A61B 5/245; G01R 33/00; G01R 33/26; A61B 5/00

(52) U.S. Cl. CPC A61B 5/245; G01R 33/0017; A61B 2562/0223; A61B 5/6803; G01R 33/26

(57) ABSTRACT

A magnetoencephalograph M1 includes: multiple optically pumped magnetometers 1A that measure a brain's magnetic field; multiple magnetic sensors for geomagnetic field cancellation 2 that measure a magnetic field; multiple magnetic sensors for active shield 3 that measure a fluctuating magnetic field; a geomagnetic field nulling coil; an active shield coil 9; a control device 5 that determines a current to generate a magnetic field for canceling the magnetic field based on measured values of the multiple magnetic sensors for geomagnetic field cancellation 2, determines a current to generate a magnetic field for canceling the fluctuating magnetic field based on measured values of the multiple magnetic sensors for active shield 3, and outputs a control signal corresponding to each of the determined currents; and a coil power supply 6 that outputs a current to each coil in response to the control signal.

### TECHNICAL FIELD

Aspects of the present invention relate to a magnetoencephalograph and a brain's magnetic field measurement method.

### BACKGROUND

In the related art, as a magnetoencephalograph, a superconducting quantum interference device (SQUID) has been used to measure small magnetism. In recent years, a magnetoencephalograph using a optically pumped magnetometer instead of the SQUID has been studied. The optically pumped magnetometer measures small magnetic fields by using the spin polarization of alkali metal atoms excited by optical pumping. For example, Japanese Patent No. 5823195 discloses a magnetoencephalograph using an optical pumped magnetometer.

### SUMMARY

In order to avoid the influence of magnetic noise stronger than the brain's magnetic field, the measurement by the magnetoencephalograph is performed in a magnetic shield room that shields the magnetic noise. However, the installation of the magnetic shield room is restricted from the viewpoint of weight, price, and the like.

Aspects of the present invention have been made in view of the above circumstances, and it is an object of the present invention to provide a magnetoencephalograph and a brain's magnetic field measurement method capable of performing measurement with high accuracy without using a magnetic shield room.

A magnetoencephalograph according to one aspect of the present invention includes: multiple optically pumped magnetometers that measure a brain's magnetic field; multiple magnetic sensors for geomagnetic field cancellation that measure a magnetic field relevant to geomagnetism at a position of each of the multiple optically pumped magnetometers; multiple magnetic sensors for active shield that measure a fluctuating magnetic field at the position of each of the multiple optically pumped magnetometers; a geomagnetic field nulling coil for canceling the magnetic field relevant to the geomagnetism; an active shield coil for canceling the fluctuating magnetic field; a control device that determines a current for the geomagnetic field nulling coil so as to generate a magnetic field for canceling the magnetic field relevant to the geomagnetism based on measured values of the multiple magnetic sensors for geomagnetic field cancellation, determines a current for the active shield coil so as to generate a magnetic field for canceling the fluctuating magnetic field based on measured values of the multiple magnetic sensors for active shield, and outputs a control signal corresponding to each of the determined currents; and a coil power supply that outputs a current to each of the geomagnetic field nulling coil and the active shield coil in response to the control signal output from the control device.

In the magnetoencephalograph according to one aspect of the present invention, the magnetic field relevant to the geomagnetism and the fluctuating magnetic field at the position of each of the multiple optically pumped magnetometers for measuring the brain's magnetic field are measured. Then, in this magnetoencephalograph, the current for the geomagnetic field nulling coil is determined so as to generate a magnetic field for canceling the magnetic field relevant to the geomagnetism based on the multiple measured values of the magnetic field relevant to the geomagnetism, the current for the active shield coil is determined so as to generate a magnetic field for canceling the fluctuating magnetic field based on the multiple measured values of the fluctuating magnetic field, and the control signal corresponding to each of the determined currents is output. Then, when the current corresponding to the control signal is output to each of the geomagnetic field nulling coil and the active shield coil, a magnetic field is generated in each coil. At the positions of the multiple optically pumped magnetometers, the magnetic field relevant to the geomagnetism is canceled by the magnetic field generated in the geomagnetic field nulling coil, and the fluctuating magnetic field is canceled by the magnetic field generated in the active shield coil. Therefore, since the magnetic field relevant to the geomagnetism and the fluctuating magnetic field at the positions of the multiple optically pumped magnetometers are canceled, the multiple optically pumped magnetometers can measure the brain's magnetic field in a state in which the influence of the magnetic field relevant to the geomagnetism and the influence of the fluctuating magnetic field are avoided. According to such a magnetoencephalograph, the brain's magnetic field can be measured with high accuracy without using the magnetic shield room.

The geomagnetic field nulling coil may include a geomagnetism nulling coil for canceling a magnetic field of the geomagnetism and a gradient magnetic field nulling coil for canceling a gradient magnetic field of the geomagnetism. The control device may determine a current for the geomagnetism nulling coil so that an average value of the measured values of the multiple magnetic sensors for geomagnetic field cancellation approaches zero and determine a current for the gradient magnetic field nulling coil so that a deviation from the average value of the measured values of the multiple magnetic sensors for geomagnetic field cancellation is minimized. In such a configuration, uniform magnetic field cancellation (0th-order cancellation) is performed by controlling the current for the geomagnetism nulling coil, and gradient magnetic field cancellation (first-order cancellation) considering the difference between the positions of the optically pumped magnetometers is performed by controlling the current for the gradient magnetic field nulling coil. In this manner, since the geomagnetism and the gradient magnetic field of the geomagnetism are canceled stepwise, the magnetic field relevant to the geomagnetism can be canceled with high accuracy.

Each of the geomagnetism nulling coil and the gradient magnetic field nulling coil may be a pair of coils arranged with the multiple optically pumped magnetometers interposed therebetween. According to such a configuration, the magnetic field relevant to the geomagnetism at the positions of the multiple optically pumped magnetometers interposed between a pair of geomagnetism nulling coils and between a pair of gradient magnetic field nulling coils is effectively canceled. In this manner, the magnetic field relevant to the geomagnetism can be appropriately canceled by a simple configuration.

The geomagnetic field nulling coil may include coil systems, which are arranged so as to be perpendicular to each other and surround each of the multiple optically pumped magnetometers and which are able to apply magnetic fields in three directions perpendicular to each other, for each of the multiple optically pumped magnetometers, and the control device may determine currents for the coil systems for each of the multiple optically pumped magnetometers so that the measured values of the multiple magnetic sensors for geomagnetic field cancellation approach zero. According to such a configuration, the coil systems are arranged for each of the multiple optically pumped magnetometers so as to correspond to the components of the static magnetic field in the three directions (x axis, y axis, and z axis). Then, by controlling the current for each of the coil systems, a magnetic field that cancels each of the x-axis direction component, the y-axis direction component, and the z-axis direction component of the magnetic field relevant to the geomagnetism is generated for each of the multiple optically pumped magnetometers, and the magnetic field relevant to the geomagnetism is canceled in the three directions. Therefore, since the current can be finely controlled for each of the multiple optically pumped magnetometers, the cancellation accuracy of the magnetic field relevant to the geomagnetism is improved. In addition, since only the magnetic field relevant to the geomagnetism in a region relevant to the operation of the multiple optically pumped magnetometers is canceled, it is possible to suppress an increase in power consumption due to unnecessary cancellation.

The control device may determine a current for the active shield coil so that an average value of the measured values of the multiple magnetic sensors for active shield approaches zero. According to such a configuration, the fluctuating magnetic field at the positions of the multiple optically pumped magnetometers is effectively canceled by controlling the current for the active shield coil. In this manner, the fluctuating magnetic field can be appropriately canceled by a simple configuration.

The multiple optically pumped magnetometers may be axial gradiometers having a measurement region and a reference region in a direction perpendicular to a scalp and coaxially. According to such a configuration, since the influence of common mode noise is shown in each of the output result of the measurement region and the output result of the reference region, the common mode noise can be removed by acquiring the difference between the output results of both. Therefore, the measurement accuracy of the brain's magnetic field is improved.

The multiple optically pumped magnetometers, the multiple magnetic sensors for geomagnetic field cancellation, and the multiple magnetic sensors for active shield may be fixed to a non-magnetic frame which is a helmet-type frame attached to a head of a subject and whose relative permeability is close to 1 so that a magnetic field distribution is not affected. According to such a configuration, the non-magnetic frame attached to the head and each sensor fixed to the non-magnetic frame move according to the movement of the head of the subject. Therefore, even when the head of the subject moves, it is possible to appropriately cancel the magnetic field relevant to the geomagnetism and the fluctuating magnetic field at the positions of the multiple optically pumped magnetometers and measure the brain's magnetic field.

The magnetoencephalograph according to one aspect of the present invention may further include an electromagnetic shield for shielding high-frequency electromagnetic noise. According to such a configuration, it is possible to prevent high-frequency electromagnetic noise, which cannot be measured by the magnetoencephalograph, from entering the multiple optically pumped magnetometers. As a result, the multiple optically pumped magnetometers can be stably operated.

A brain's magnetic field measurement method according to another aspect of the present invention includes: measuring a magnetic field relevant to geomagnetism at a position of each of multiple optically pumped magnetometers; determining a current for a geomagnetic field nulling coil so as to generate a magnetic field for canceling the magnetic field relevant to the geomagnetism based on multiple measured values of the magnetic field relevant to the geomagnetism and outputting a control signal for geomagnetic field cancellation corresponding to the determined current; outputting a current to the geomagnetic field nulling coil in response to the control signal for geomagnetic field cancellation; measuring a fluctuating magnetic field at the position of each of the multiple optically pumped magnetometers; determining a current for an active shield coil so as to generate a magnetic field for canceling the fluctuating magnetic field based on multiple measured values of the fluctuating magnetic field and outputting a control signal for fluctuating magnetic field cancellation corresponding to the determined current; outputting a current to the active shield coil in response to the control signal for fluctuating magnetic field cancellation; and measuring a brain's magnetic field with the multiple optically pumped magnetometers.

In the brain's magnetic field measurement method according to another aspect of the present invention, the magnetic field relevant to the geomagnetism and the fluctuating magnetic field at the position of each of the multiple optically pumped magnetometers for measuring the brain's magnetic field are measured. Then, in the brain's magnetic field measurement method, the current for the geomagnetic field nulling coil is determined so as to generate a magnetic field for canceling the magnetic field relevant to the geomagnetism based on the multiple measured values of the magnetic field relevant to the geomagnetism, and the control signal corresponding to the determined current is output. Then, when the current corresponding to the control signal is output to the geomagnetic field nulling coil, a magnetic field is generated in the geomagnetic field nulling coil. At the positions of the multiple optically pumped magnetometers, the magnetic field relevant to the geomagnetism is canceled by the magnetic field generated in the geomagnetic field nulling coil. In addition, the current for the active shield coil is determined so as to generate a magnetic field for canceling the fluctuating magnetic field based on the multiple measured values of the fluctuating magnetic field, and the control signal corresponding to the determined current is output. Then, when the current corresponding to the control signal is output to the active shield coil, a magnetic field is generated in the active shield coil. At the positions of the multiple optically pumped magnetometers, the fluctuating magnetic field is canceled by the magnetic field generated in the active shield coil. As a result, since the magnetic field relevant to the geomagnetism and the fluctuating magnetic field at the positions of the multiple optically pumped magnetometers are canceled, the multiple optically pumped magnetometers can measure the brain's magnetic field in a state in which the influence of the magnetic field relevant to the geomagnetism and the influence of the fluctuating magnetic field are avoided. According to such a brain's magnetic field measurement method, the brain's magnetic field can be measured with high accuracy without using the magnetic shield room.

Determining the current for the geomagnetic field nulling coil so as to generate a magnetic field for canceling the magnetic field relevant to the geomagnetism may include: determining a current for a geomagnetism nulling coil forming the geomagnetic field nulling coil so that an average value of the multiple measured values of the magnetic field relevant to the geomagnetism approaches zero; and determining a current for a gradient magnetic field nulling coil forming the geomagnetic field nulling coil so that a deviation from the average value of the multiple measured values of the magnetic field relevant to the geomagnetism is minimized. In such a method, uniform magnetic field cancellation (0th-order cancellation) is performed by controlling the current for the geomagnetism nulling coil, and gradient magnetic field cancellation (first-order cancellation) considering the difference between the positions of the optically pumped magnetometers is performed by controlling the current for the gradient magnetic field nulling coil. In this manner, since the geomagnetism and the gradient magnetic field of the geomagnetism are canceled stepwise, the magnetic field relevant to the geomagnetism can be canceled with high accuracy.

Determining the current for the geomagnetic field nulling coil so as to generate a magnetic field for canceling the magnetic field relevant to the geomagnetism may include determining currents for coil systems, which are arranged so as to be perpendicular to each other and surround each of the multiple optically pumped magnetometers, so that the multiple measured values of the magnetic field relevant to the geomagnetism approach zero. According to such a method, the coil systems are arranged for each of the multiple optically pumped magnetometers so as to correspond to the components of the static magnetic field in the three directions (x axis, y axis, and z axis). Then, by controlling the current for each of the coil systems, a magnetic field that cancels each of the x-axis direction component, the y-axis direction component, and the z-axis direction component of the magnetic field relevant to the geomagnetism is generated for each of the multiple optically pumped magnetometers, and the magnetic field relevant to the geomagnetism is canceled in the three directions. Therefore, since the current can be finely controlled for each of the multiple optically pumped magnetometers, the cancellation accuracy of the magnetic field relevant to the geomagnetism is improved. In addition, since only the magnetic field relevant to the geomagnetism in a region relevant to the operation of the multiple optically pumped magnetometers is canceled, it is possible to suppress an increase in power consumption due to unnecessary cancellation.

According to aspects of the present invention, it is possible to provide a magnetoencephalograph and a brain's magnetic field measurement method capable of performing measurement with high accuracy without using a magnetic shield room.

### BRIEF DESCRIPTION OF THE DRAWINGS

FIG. 1 is a schematic diagram showing the configuration of a magnetoencephalograph according to an embodiment.

FIG. 2 is a flowchart showing the operation of the magnetoencephalograph according to the embodiment.

FIG. 3 is a schematic diagram showing the configuration of a magnetoencephalograph according to another embodiment.

FIG. 4 is a diagram showing the arrangement of a coil system.

FIG. 5 is a flowchart showing the operation of the magnetoencephalograph according to another embodiment.

### DETAILED DESCRIPTION

Hereinafter, an embodiment for carrying out the present invention will be described in detail with reference to the accompanying diagrams. In the description of the diagrams, the same elements are denoted by the same reference numerals, and the repeated description thereof will be omitted.

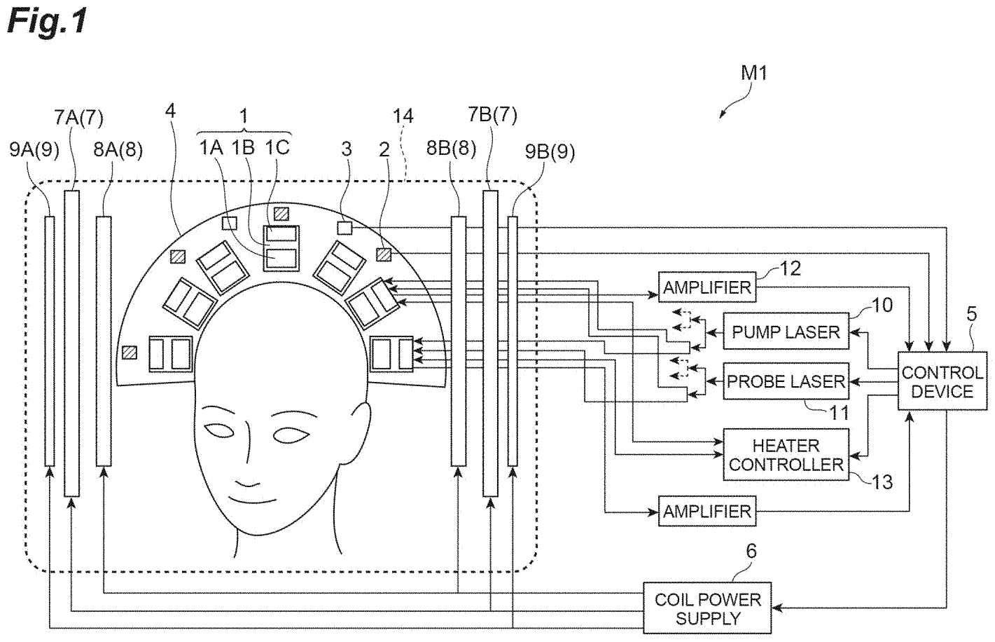

FIG. 1 is a schematic diagram showing the configuration of a magnetoencephalograph M1 according to an embodiment. The magnetoencephalograph M1 is an apparatus that measures a magnetic field of the brain by using optically pumped magnetometers while generating a magnetic field that cancels magnetic noise. The magnetoencephalograph M1 includes multiple optically pumped magnetometer (OPM) modules 1, multiple magnetic sensors for geomagnetic field cancellation 2, multiple magnetic sensors for active shield 3, a non-magnetic frame 4, a control device 5, a coil power supply 6, a pair of geomagnetism nulling coils 7, a pair of gradient magnetic field nulling coils 8 (geomagnetic field nulling coils), a pair of active shield coils 9, a pump laser 10, a probe laser 11, an amplifier 12, a heater controller 13, and an electromagnetic shield 14.

Each OPM module 1 includes an optically pumped magnetometer 1A, a heat insulating material 1B, and a read circuit 1C. The multiple OPM modules 1 are arranged at predetermined intervals along the scalp, for example.

The optically pumped magnetometer 1A is a sensor that measures a brain's magnetic field by using optical pumping, and has a sensitivity of, for example, about 10 fT to 10 pT. The heat insulating material 1B prevents heat transfer of the optically pumped magnetometer 1A heated to 180° by a heater (not shown). The read circuit 1C is a circuit for acquiring the detection result of the optically pumped magnetometer 1A. The optically pumped magnetometer 1A comprising a cell containing alkali metal vapor is irradiated by a pump light to excite the alkali metal. The excited alkali metal is in a spin polarization state, and when this receives magnetism, the inclination of the spin polarization axis of the alkali metal atom changes according to the magnetism. The inclination of the spin polarization axis is detected by probe light emitted separately from the pump light. The read circuit 1C receives probe light passing through the alkali metal vapor by a photodiode and acquires the detection result. The read circuit 1C outputs the detection result to the amplifier 12.

The optically pumped magnetometer 1A may be, for example, an axial gradiometer. The axial gradiometer has a measurement region and a reference region in a direction perpendicular to the scalp (measurement portion) of the subject and coaxially. The measurement region is, for example, a portion closest to the scalp of the subject among portions where the axial gradiometer measures the brain's magnetic field. The reference region is, for example, a portion away from the measurement region by a predetermined distance (for example, 3 cm) in a direction away from the scalp of the subject, among portions where the axial gradiometer measures the brain's magnetic field. The axial gradiometer outputs the respective measurement results in the measurement region and the reference region to the amplifier 12. Here, when common mode noise is included, its influence is shown in each of the output result of the measurement region and the output result of the reference region. Common mode noise is removed by acquiring the difference between the output result of the measurement region and the output result of the reference region. By removing the common mode noise, the optically pumped magnetometer 1A can obtain a sensitivity of about 10 fT/√ Hz, for example, when performing measurement in a magnetic noise environment of 1 pT.

The magnetic sensor for geomagnetic field cancellation 2 is a sensor that measures a magnetic field relevant to the geomagnetism at a position corresponding to the optically pumped magnetometer 1A, and is, for example, a flux gate sensor having a sensitivity of about 1 nT to 100 μT. The position corresponding to the optically pumped magnetometer 1A is a position around (near) the region where the optically pumped magnetometer 1A is arranged. The magnetic sensor for geomagnetic field cancellation 2 may be provided so as to correspond to the optically pumped magnetometer 1A in a one-to-one manner, or may be provided so as to correspond in a one-to-many manner (one magnetic sensor for geomagnetic field cancellation 2 for multiple optically pumped magnetometers 1A). The magnetic sensor for geomagnetic field cancellation 2 measures, for example, geomagnetism and a gradient magnetic field of the geomagnetism (hereinafter, simply referred to as “gradient magnetic field”) as magnetic fields relevant to the geomagnetism, and outputs the measured value to the control device 5. The measured value of the magnetic sensor for geomagnetic field cancellation 2 can be expressed by a vector having a direction and a magnitude. The magnetic sensor for geomagnetic field cancellation 2 may continuously perform measurement and output at predetermined time intervals.

The magnetic sensor for active shield 3 is a sensor that measures a fluctuating magnetic field at a position corresponding to the optically pumped magnetometer 1A, and is, for example, a optically pumped magnetometer having a sensitivity of about 100 fT to 10 nT in a frequency band of several hundred Hz or less and different from the optically pumped magnetometer 1A. The position corresponding to the optically pumped magnetometer 1A is a position around (near) the region where the optically pumped magnetometer 1A is arranged. The magnetic sensor for active shield 3 may be provided so as to correspond to the optically pumped magnetometer 1A in a one-to-one manner, or may be provided so as to correspond in a one-to-many manner (one magnetic sensor for active shield 3 for the multiple optically pumped magnetometers 1A). The magnetic sensor for active shield 3 measures a magnetic field of a noise (AC) component of, for example, 200 Hz or less as a fluctuating magnetic field, and outputs the measured value to the control device 5. The measured value of the magnetic sensor for active shield 3 can be expressed by a vector having a direction and a magnitude.

The non-magnetic frame 4 is a frame that covers the entire scalp of the subject whose brain's magnetic field is to be measured, and is formed of a non-magnetic material such as graphite whose relative permeability is close to 1 and accordingly does not affect the magnetic field distribution. The non-magnetic frame 4 can be, for example, a helmet-type frame that surrounds the entire scalp of the subject and is attached to the head of the subject. The multiple optically pumped magnetometers 1A are fixed to the non-magnetic frame 4 so as to be close to the scalp of the subject. In addition, the magnetic sensor for geomagnetic field cancellation 2 is fixed to the non-magnetic frame 4 so that a magnetic field relevant to the geomagnetism at the position of each of the multiple optically pumped magnetometers 1A can be measured, and the magnetic sensor for active shield 3 is fixed to the non-magnetic frame 4 so that a fluctuating magnetic field at the position of each of the multiple optically pumped magnetometers 1A can be measured. Since a change in the magnetic field strength according to the position of the fluctuating magnetic field is smaller than that in the case of the static magnetic field, a smaller number of magnetic sensors for active shield 3 than the number of magnetic sensors for geomagnetic field cancellation 2 may be fixed to the non-magnetic frame 4.

The control device 5 is a device that determines currents for various coils based on the measured values output from the magnetic sensor for geomagnetic field cancellation 2 and the magnetic sensor for active shield 3, and outputs a control signal for outputting each of the currents to the coil power supply 6. Based on the measured values of the multiple magnetic sensors for geomagnetic field cancellation 2, the control device 5 determines a current for the geomagnetism nulling coil 7 and the gradient magnetic field nulling coil 8, which are geomagnetic field nulling coils, so as to generate a magnetic field for canceling a magnetic field relevant to the geomagnetism. In addition, based on the measured values of the multiple magnetic sensors for active shield 3, the control device 5 determines a current for the active shield coil 9 so as to generate a magnetic field for canceling a fluctuating magnetic field. The control device 5 outputs a control signal corresponding to the determined current to the coil power supply 6.

Specifically, the control device 5 determines a current for the geomagnetism nulling coil 7 so that the average value of the measured values of the multiple magnetic sensors for geomagnetic field cancellation 2 approaches zero (as a result, a magnetic field opposite to the geomagnetism at the position of the optically pumped magnetometer 1A and having approximately the same magnitude as the geomagnetism is generated). The control device 5 outputs a control signal (control signal for static magnetic field cancellation) corresponding to the determined current of the geomagnetism nulling coil 7 to the coil power supply 6.

In addition, the control device 5 determines a current for the gradient magnetic field nulling coil 8 so that the deviation from the average value of the measured values of the multiple magnetic sensors for geomagnetic field cancellation 2 is minimized (as a result, a magnetic field opposite to the gradient magnetic field at the position of the optically pumped magnetometer 1A and having approximately the same magnitude as the gradient magnetic field is generated). The control device 5 outputs a control signal (control signal for static magnetic field cancellation) corresponding to the determined current of the gradient magnetic field nulling coil 8 to the coil power supply 6.

In addition, the control device 5 determines a current for the active shield coil 9 so that the average value of the measured values of the multiple magnetic sensors for active shield 3 approaches zero (as a result, a magnetic field opposite to the fluctuating magnetic field at the position of the optically pumped magnetometer 1A and having approximately the same magnitude as the fluctuating magnetic field is generated). The control device 5 outputs a control signal (control signal for fluctuating magnetic field cancellation) corresponding to the determined current of the active shield coil 9 to the coil power supply 6.

In addition, the control device 5 obtains information regarding the magnetism detected by the optically pumped magnetometer 1A by using the signal output from the amplifier 12. When the optically pumped magnetometer 1A is an axial gradiometer, the control device 5 may remove the common mode noise by acquiring the difference between the output result of the measurement region and the output result of the reference region. In addition, the control device 5 may control operations such as the emission timing and the emission time of the pump laser 10 and the probe laser 11.

The control device 5 is physically configured to include a memory such as a RAM and a ROM, a processor (arithmetic circuit) such as a CPU, a communication interface, and a storage unit such as a hard disk. Examples of the control device 5 include a personal computer, a cloud server, a smartphone, and a tablet terminal. The control device 5 functions by executing a program stored in the memory on the CPU of the computer system.

The coil power supply 6 outputs a predetermined current to each of the geomagnetism nulling coil 7, the gradient magnetic field nulling coil 8, and the active shield coil 9 in response to the control signal output from the control device 5. Specifically, the coil power supply 6 outputs a current to the geomagnetism nulling coil 7 in response to the control signal relevant to the geomagnetism nulling coil 7. The coil power supply 6 outputs a current to the gradient magnetic field nulling coil 8 in response to the control signal relevant to the gradient magnetic field nulling coil 8. The coil power supply 6 outputs a current to the active shield coil 9 in response to the control signal relevant to the active shield coil 9.

The geomagnetism nulling coil 7 is a coil for canceling the magnetic field of the geomagnetism among the magnetic fields relevant to the geomagnetism at the position of the optically pumped magnetometer 1A. The geomagnetism nulling coil 7 generates a magnetic field according to the current supplied from the coil power supply 6 to cancel the geomagnetism. The geomagnetism nulling coil 7 has, for example, a pair of geomagnetism nulling coils 7A and 7B. The pair of geomagnetism nulling coils 7A and 7B are arranged with the optically pumped magnetometer 1A interposed therebetween (for example, on the left and right of the subject). The pair of geomagnetism nulling coils 7A and 7B generate a magnetic field, which is opposite to the geomagnetism at the position of the optically pumped magnetometer 1A and has approximately the same magnitude as the geomagnetism, according to the current supplied from the coil power supply 6. The direction of the magnetic field is, for example, from one geomagnetism nulling coil 7A to the other geomagnetism nulling coil 7B. The geomagnetism at the position of the optically pumped magnetometer 1A is canceled by a magnetic field generated by the geomagnetism nulling coil 7, the magnetic field being opposite to the geomagnetism and having approximately the same magnitude as the geomagnetism. In this manner, the geomagnetism nulling coil 7 cancels the geomagnetism at the position of the optically pumped magnetometer 1A.

The gradient magnetic field nulling coil 8 is a coil for canceling the gradient magnetic field among the magnetic fields relevant to the geomagnetism at the position of the optically pumped magnetometer 1A. The gradient magnetic field nulling coil 8 generates a magnetic field according to the current supplied from the coil power supply 6 to cancel the gradient magnetic field. The gradient magnetic field nulling coil 8 has, for example, a pair of gradient magnetic field nulling coils 8A and 8B. The pair of gradient magnetic field nulling coils 8A and 8B are arranged with the optically pumped magnetometer 1A interposed therebetween (for example, on the left and right of the subject). The pair of gradient magnetic field nulling coils 8A and 8B generate a magnetic field, which is opposite to the gradient magnetic field at the position of the optically pumped magnetometer 1A and has approximately the same magnitude as the gradient magnetic field, according to the current supplied from the coil power supply 6. The direction of the magnetic field is, for example, from one gradient magnetic field nulling coil 8A to the other gradient magnetic field nulling coil 8B. The gradient magnetic field at the position of the optically pumped magnetometer 1A is canceled by a magnetic field generated by the gradient magnetic field nulling coil 8, the magnetic field being opposite to the gradient magnetic field and having approximately the same magnitude as the gradient magnetic field. In this manner, the gradient magnetic field nulling coil 8 cancels the gradient magnetic field at the position of the optically pumped magnetometer 1A.

The active shield coil 9 is a coil for canceling the fluctuating magnetic field at the position of the optically pumped magnetometer 1A. The active shield coil 9 generates a magnetic field according to the current supplied from the coil power supply 6 to cancel the fluctuating magnetic field. The active shield coil 9 has, for example, a pair of active shield coils 9A and 9B. The pair of active shield coils 9A and 9B are arranged with the optically pumped magnetometer 1A interposed therebetween (for example, on the left and right of the subject). The pair of active shield coils 9A and 9B generate a magnetic field, which is opposite to the fluctuating magnetic field at the position of the optically pumped magnetometer 1A and has approximately the same magnitude as the fluctuating magnetic field, according to the current supplied from the coil power supply 6. The direction of the magnetic field is, for example, from one active shield coil 9A to the other active shield coil 9B. The fluctuating magnetic field at the position of the optically pumped magnetometer 1A is canceled by a magnetic field generated by the active shield coil 9, the magnetic field being opposite to the fluctuating magnetic field and having approximately the same magnitude as the fluctuating magnetic field. In this manner, the active shield coil 9 cancels the fluctuating magnetic field at the position of the optically pumped magnetometer 1A.

The pump laser 10 is a laser device that generates pump light. The pump light emitted from the pump laser 10 is incident on each of the multiple optically pumped magnetometers 1A by fiber branching.

The probe laser 11 is a laser device that generates probe light. The probe light emitted from the probe laser 11 is incident on each of the multiple optically pumped magnetometers 1A by fiber branching.

The amplifier 12 is a device or circuit that amplifies an output result signal from the OPM module 1 (specifically, the read circuit 1C) and outputs the signal to the control device 5.

The heater controller 13 is a temperature adjusting device connected to a heater (not shown) for heating the cell of the optically pumped magnetometer 1A and a thermocouple (not shown) for measuring the temperature of the cell. The heater controller 13 adjusts the temperature of each cell by receiving the temperature information of the cell from the thermocouple and adjusting the heating of the heater based on the temperature information.

The electromagnetic shield 14 is a shield member for shielding high-frequency (for example, 10 kHz or higher) electromagnetic noise.

For example, the electromagnetic shield 14 is formed of a mesh woven with metal threads, a non-magnetic metal plate such as aluminum, or the like. The electromagnetic shield 14 is arranged so as to surround the optically pumped magnetometer 1A, the magnetic sensor for geomagnetic field cancellation 2, the magnetic sensor for active shield 3, the non-magnetic frame 4, the geomagnetism nulling coil 7, the gradient magnetic field nulling coil 8, and the active shield coil 9.

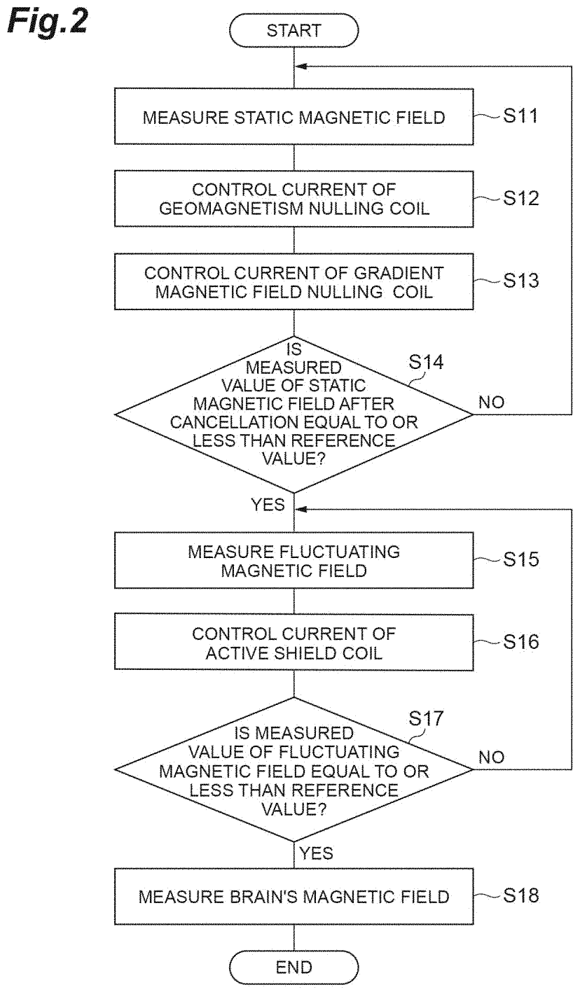

Next, a brain's magnetic field measurement method using the magnetoencephalograph M1 according to the embodiment will be described with reference to FIG. 2. FIG. 2 is a flowchart showing the operation of the magnetoencephalograph M1.

The magnetic sensor for geomagnetic field cancellation 2 measures a magnetic field relevant to the geomagnetism, which is a static magnetic field (step S11). The magnetic sensor for geomagnetic field cancellation 2 measures the geomagnetism and the gradient magnetic field at each position of the optically pumped magnetometer 1A, and outputs the measured values to the control device 5.

The control device 5 and the coil power supply 6 control a current for the geomagnetism nulling coil 7 (step S12). The control device 5 determines a current for the geomagnetism nulling coil 7 based on the measured value of the magnetic sensor for geomagnetic field cancellation 2 so that a magnetic field opposite to the geomagnetism at the position of the optically pumped magnetometer 1A and having approximately the same magnitude as the geomagnetism is generated. More specifically, the control device 5 determines a current for the geomagnetism nulling coil 7 so that the average value of the measured values of the multiple magnetic sensors for geomagnetic field cancellation 2 approaches zero, for example. The control device 5 outputs a control signal corresponding to the determined current to the coil power supply 6. The coil power supply 6 outputs a predetermined current to the geomagnetism nulling coil 7 in response to the control signal output from the control device 5. The geomagnetism nulling coil 7 generates a magnetic field according to the current supplied from the coil power supply 6. The geomagnetism at the position of the optically pumped magnetometer 1A is canceled by a magnetic field generated by the geomagnetism nulling coil 7, the magnetic field being opposite to the geomagnetism and having approximately the same magnitude as the geomagnetism.

The control device 5 and the coil power supply 6 control a current for the gradient magnetic field nulling coil 8 (step S13). The control device 5 determines a current for the gradient magnetic field nulling coil 8 based on the measured value of the magnetic sensor for geomagnetic field cancellation 2 so that a magnetic field opposite to the gradient magnetic field at the position of the optically pumped magnetometer 1A and having approximately the same magnitude as the gradient magnetic field is generated. More specifically, the control device 5 determines a current for the gradient magnetic field nulling coil 8 so that the deviation from the average value of the measured values of the multiple magnetic sensors for geomagnetic field cancellation 2 is minimized, for example. The control device 5 outputs a control signal corresponding to the determined current to the coil power supply 6. The coil power supply 6 outputs a predetermined current to the gradient magnetic field nulling coil 8 in response to the control signal output from the control device 5. The gradient magnetic field nulling coil 8 generates a magnetic field according to the current supplied from the coil power supply 6. The gradient magnetic field at the position of the optically pumped magnetometer 1A is canceled by a magnetic field generated by the gradient magnetic field nulling coil 8, the magnetic field being opposite to the gradient magnetic field and having approximately the same magnitude as the gradient magnetic field.

The control device 5 determines whether or not the measured value of the static magnetic field (magnetic field relevant to the geomagnetism) after the cancellation is equal to or less than the reference value (step S14). The measured value of the static magnetic field after the cancellation is a value measured by the magnetic sensors for geomagnetic field cancellation 2 after the static magnetic field is canceled by the geomagnetism nulling coil 7 and the gradient magnetic field nulling coil 8. The reference value is the magnitude of the magnetic field in which the optically pumped magnetometer 1A normally operates, and can be set to, for example, 1 nT. If the measured value of the static magnetic field is not equal to or less than the reference value (“NO” in step S14), the process returns to step S11. If the measured value of the static magnetic field is equal to or less than the reference value (“YES” in step S14), the process proceeds to step S15.

The magnetic sensor for active shield 3 measures a fluctuating magnetic field (step S15). The magnetic sensor for active shield 3 measures a fluctuating magnetic field at each position of the optically pumped magnetometer 1A and outputs the measured value to the control device 5.

The control device 5 and the coil power supply 6 control a current for the active shield coil 9 (step S16). The control device 5 determines a current for the active shield coil 9 based on the measured value of the magnetic sensor for active shield 3 so that a magnetic field opposite to the fluctuating magnetic field at the position of the optically pumped magnetometer 1A and having approximately the same magnitude as the fluctuating magnetic field is generated. More specifically, the control device 5 determines a current for the active shield coil 9 so that the average value of the measured values of the multiple magnetic sensors for active shield 3 approaches zero, for example. The control device 5 outputs a control signal corresponding to the determined current to the coil power supply 6. The coil power supply 6 outputs a predetermined current to the active shield coil 9 in response to the control signal output from the control device 5. The active shield coil 9 generates a magnetic field according to the current supplied from the coil power supply 6. The fluctuating magnetic field at the position of the optically pumped magnetometer 1A is canceled by a magnetic field generated by the active shield coil 9, the magnetic field being opposite to the fluctuating magnetic field and having approximately the same magnitude as the fluctuating magnetic field.

The control device 5 determines whether or not the measured value of the fluctuating magnetic field after the cancellation is equal to or less than the reference value (step S17). The measured value of the fluctuating magnetic field after the cancellation is a value measured by the magnetic sensor for active shield 3 after the fluctuating magnetic field is canceled by the active shield coil 9. The reference value is a noise level at which the brain's magnetic field can be measured, and can be set to, for example, 1 pT. If the measured value of the fluctuating magnetic field is not less than or equal to the reference value (“NO” in step S17), the process returns to step S15. If the measured value of the fluctuating magnetic field is equal to or less than the reference value (“YES” in step S17), the process proceeds to step S18.

The optically pumped magnetometer 1A measures a brain's magnetic field (step S18). Since the static magnetic field (magnetic field relevant to the geomagnetism) and the fluctuating magnetic field at the position of the optically pumped magnetometer 1A are canceled so as to be equal to or less than a predetermined reference value, the optically pumped magnetometer 1A can measure the brain's magnetic field in a state in which the influence of the static magnetic field (magnetic field relevant to the geomagnetism) and the influence of the fluctuating magnetic field are avoided.

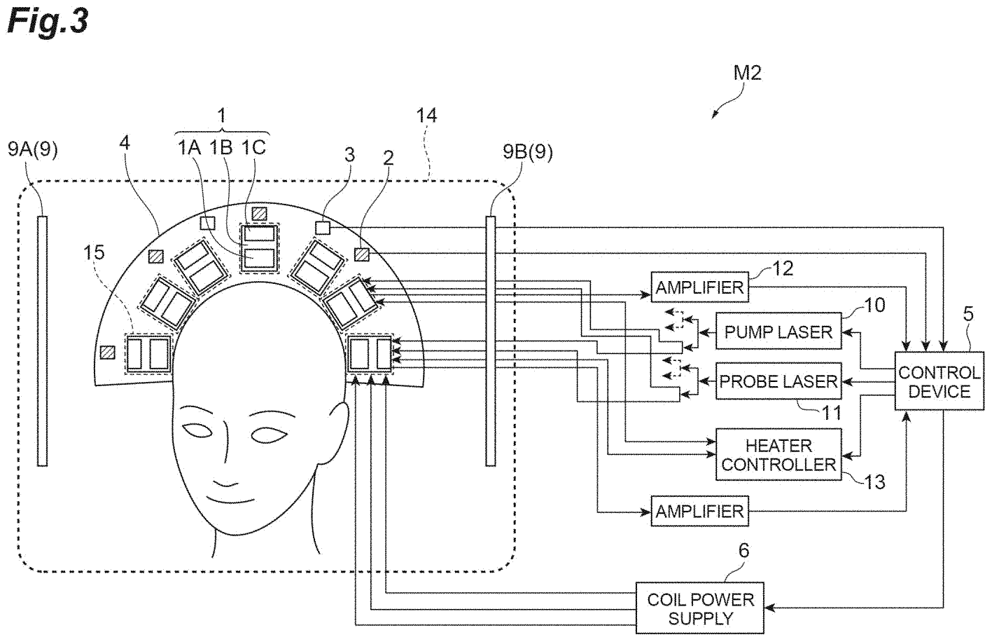

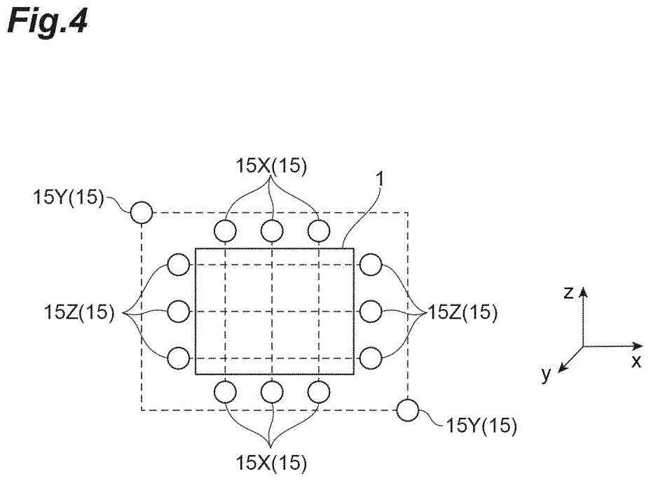

FIG. 3 is a schematic diagram showing the configuration of a magnetoencephalograph M2 according to another embodiment. Similar to the magnetoencephalograph M1, the magnetoencephalograph M2 is an apparatus that measures a magnetic field of the brain by using optically magnetometers while generating a magnetic field that cancels magnetic noise. The magnetoencephalograph M2 includes an OPM module 1, a magnetic sensor for geomagnetic field cancellation 2, a magnetic sensor for active shield 3, a non-magnetic frame 4, a control device 5, a coil power supply 6, an active shield coil 9, a pump laser 10, a probe laser 11, an amplifier 12, a heater controller 13, an electromagnetic shield 14, and a coil system 15 (geomagnetic field nulling coil). In the magnetoencephalograph M2, instead of the geomagnetism nulling coil 7 and the gradient magnetic field nulling coil 8 of the magnetoencephalograph M1, the coil system 15 is arranged for each OPM module 1 (optically pumped magnetometer 1A). Here, the arrangement of the coil system 15 will be described with reference to FIG. 4.

FIG. 4 is a diagram showing the arrangement of the coil system 15 according to the magnetoencephalograph M2. The coil system 15 includes coil systems, which are arranged so as to be perpendicular to each other and which can apply magnetic fields in three directions perpendicular to each other (for example, a three-axis Helmholtz coil or a planar coil system). Specifically, the coil system 15 includes coil systems 15X, 15Y, and 15Z. In FIG. 4, the coil systems 15X, 15Y, and 15Z are arranged as shown by dotted lines with respect to the OPM module 1. In this manner, the coil systems 15X, 15Y, and 15Z are arranged so as to be perpendicular to each other and surround each OPM module 1 (optically pumped magnetometer 1A). The coil system 15X is a coil for canceling the component of the magnetic field relevant to the geomagnetism in the x-axis direction shown in FIG. 4. Similarly, the coil systems 15Y and 15Z are coils for canceling the components of the magnetic field relevant to the geomagnetism in the y-axis direction and the z-axis direction, respectively.

Returning to FIG. 3, the magnetoencephalograph M2 will be described focusing on only the differences from the magnetoencephalograph Ml. The control device 5 determines currents for the coil systems 15X, 15Y, and 15Z for each of the multiple optically pumped magnetometers 1A so that the measured values of the multiple magnetic sensors for geomagnetic field cancellation 2 approach zero. The control device 5 determines a current for the coil system 15X based on the measured value of the magnetic sensor for geomagnetic field cancellation 2 so that a magnetic field opposite to the x-axis direction component of the magnetic field relevant to the geomagnetism at the position of the optically pumped magnetometer 1A and having approximately the same magnitude as the x-axis direction component of the magnetic field relevant to the geomagnetism is generated. The control device 5 outputs a control signal (control signal for static magnetic field cancellation) corresponding to the determined current to the coil power supply 6. In addition, the control device 5 determines a current for the coil system 15Y based on the measured value of the magnetic sensor for geomagnetic field cancellation 2 so that a magnetic field opposite to the y-axis direction component of the magnetic field relevant to the geomagnetism at the position of the optically pumped magnetometer 1A and having approximately the same magnitude as the y-axis direction component of the magnetic field relevant to the geomagnetism is generated. The control device 5 outputs a control signal (control signal for static magnetic field cancellation) corresponding to the determined current to the coil power supply 6. In addition, the control device 5 determines a current for the coil system 15Z based on the measured value of the magnetic sensor for geomagnetic field cancellation 2 so that a magnetic field opposite to the z-axis direction component of the static magnetic field at the position of the optically pumped magnetometer 1A and having approximately the same magnitude as the z-axis direction component of the static magnetic field is generated. The control device 5 outputs a control signal (control signal for fluctuating magnetic field cancellation) corresponding to the determined current to the coil power supply 6.

The coil power supply 6 outputs a predetermined current to each of the coil systems 15X, 15Y, and 15Z in response to the control signal output from the control device 5. Specifically, the coil power supply 6 outputs a current to the coil system 15X in response to a control signal relevant to the coil system 15X. The coil power supply 6 outputs a current to the coil system 15Y in response to a control signal relevant to the coil system 15Y. The coil power supply 6 outputs a current to the coil system 15Z in response to a control signal relevant to the coil system 15Z.

The coil system 15 generates a magnetic field according to the current supplied from the coil power supply 6 to cancel the magnetic field relevant to the geomagnetism. Specifically, the coil system 15X generates a magnetic field, which is opposite to the x-axis direction component of the magnetic field relevant to the geomagnetism at the position of the optically pumped magnetometer 1A and having approximately the same magnitude as the x-axis direction component of the magnetic field relevant to the geomagnetism, according to the current supplied from the coil power supply 6. The x-axis direction component of the magnetic field relevant to the geomagnetism at the position of the optically pumped magnetometer 1A is canceled by a magnetic field generated by the coil system 15X, the magnetic field being opposite to the x-axis direction component of the magnetic field relevant to the geomagnetism and having approximately the same magnitude as the x-axis direction component of the magnetic field relevant to the geomagnetism. Similarly, the coil systems 15Y and 15Z generate magnetic fields, which are opposite to the y-axis direction component and the z-axis direction component of the magnetic field relevant to the geomagnetism at the position of the optically pumped magnetometer 1A and having approximately the same magnitude as the y-axis direction component and the z-axis direction component of the magnetic field relevant to the geomagnetism, to cancel the magnetic field relevant to the geomagnetism. In this manner, the coil system 15 cancels the magnetic field relevant to the geomagnetism at the position of the optically pumped magnetometer 1A. In addition, the information regarding the magnetism obtained by the control device 5 does not include the magnetic field generated by the coil system 15.

The electromagnetic shield 14 is arranged so as to surround the optically pumped magnetometer 1A, the magnetic sensor for geomagnetic field cancellation 2, the magnetic sensor for active shield 3, the non-magnetic frame 4, the active shield coil 9, and the coil system 15.

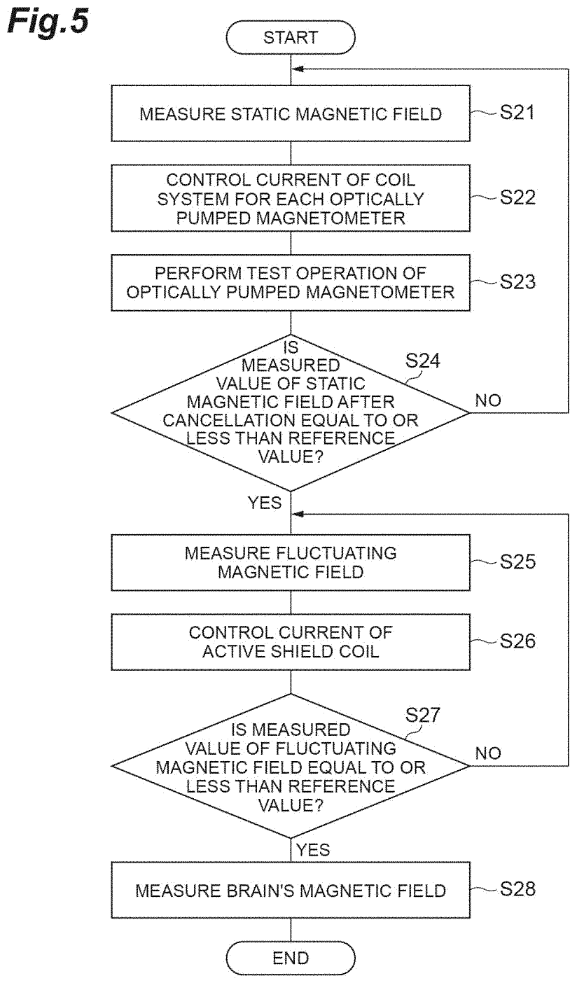

Next, a brain's magnetic field measurement method using the magnetoencephalograph M2 according to the embodiment will be described with reference to FIG. 5. FIG. 5 is a flowchart showing the operation of the magnetoencephalograph M2.

The magnetic sensor for geomagnetic field cancellation 2 measures a magnetic field relevant to the geomagnetism, which is a static magnetic field (step S21). The magnetic sensor for geomagnetic field cancellation 2 measures a magnetic field relevant to the geomagnetism including the geomagnetism and the gradient magnetic field at each position of the optically pumped magnetometer 1A, and outputs the measured value to the control device 5.

The control device 5 and the coil power supply 6 control a current for the coil system 15 for each optically pumped magnetometer 1A (step S22). The control device 5 determines a current for the coil system 15 based on the measured value of the magnetic sensor for geomagnetic field cancellation 2 so that a magnetic field opposite to each component of the magnetic field relevant to the geomagnetism in the three directions (x axis, y axis, and z axis) at the position of the optically pumped magnetometer 1A and having approximately the same magnitude as each component of the magnetic field relevant to the geomagnetism in the three directions is generated. More specifically, the control device 5 determines currents for the coil systems 15X, 15Y, and 15Z for each optically pumped magnetometer 1A so that, for example, the measured values of the multiple magnetic sensors for geomagnetic field cancellation 2 approach zero. The control device 5 outputs a control signal corresponding to the current determined for each of the coil systems 15X, 15Y, and 15Z to the coil power supply 6. The coil power supply 6 outputs a predetermined current to each of the coil systems 15X, 15Y, and 15Z in response to the control signal output from the control device 5. Each of the coil systems 15X, 15Y, and 15Z generates a magnetic field according to the current supplied from the coil power supply 6. The components of the magnetic field relevant to the geomagnetism in the three directions at the position of the optically pumped magnetometer 1A are canceled by the magnetic fields generated by the coil systems 15X, 15Y, and 15Z, the magnetic fields being opposite to the components of the magnetic field relevant to the geomagnetism in the three directions and having approximately the same magnitude as the components of the magnetic field relevant to the geomagnetism in the three directions.

A test operation of the optically pumped magnetometer 1A is performed (step S23). The optically pumped magnetometer 1A acquires the measured value of the remaining magnetic field by the test operation and outputs the measured value to the control device 5. The measured value of the magnetic field is a value measured by the optically pumped magnetometer 1A after the static magnetic field is canceled by the coil system 15.

The control device 5 determines whether or not the measured value of the magnetic field is equal to or less than a reference value (step S24). The reference value is a level at which the optically pumped magnetometer 1A operates normally, and can be set to, for example, 0.3 nT. If the measured value of the magnetic field is not equal to or less than the reference value (“NO” in step S24), the process returns to step S21. If the measured value of the magnetic field is equal to or less than the reference value (“YES” in step S24), the process proceeds to step S25.

Subsequent steps S25 to S28 are the same processes as steps S15 to S18, and accordingly the description thereof will be omitted. The magnetic sensor for active shield 3 measures a fluctuating magnetic field (step S25).

The control device 5 controls a current for the active shield coil 9 (step S26).

The control device 5 determines whether or not the measured value of the fluctuating magnetic field after the cancellation is equal to or less than the reference value (step S27). If the measured value of the fluctuating magnetic field is not equal to or less than the reference value (“NO” in step S27), the process returns to step S25. If the measured value of the fluctuating magnetic field is equal to or less than the reference value (“YES” in step S27), the process proceeds to step S28.

The optically pumped magnetometer 1A measures a brain's magnetic field (step S28).

### Operational Effects

Next, the operational effects of the magnetoencephalograph according to the above embodiment will be described.

Each of the magnetoencephalographs M1 and M2 according to the present embodiment includes: multiple optically pumped magnetometers 1A that measure a brain's magnetic field; multiple magnetic sensors for geomagnetic field cancellation 2 that measure a magnetic field relevant to geomagnetism at a position of each of the multiple optically pumped magnetometers 1A; multiple magnetic sensors for active shield 3 that measure a fluctuating magnetic field at the position of each of the multiple optically pumped magnetometers 1A; a geomagnetic field nulling coil for canceling the magnetic field relevant to the geomagnetism; the active shield coil 9 for canceling the fluctuating magnetic field; the control device 5 that determines a current for the geomagnetic field nulling coil so as to generate a magnetic field for canceling the magnetic field relevant to the geomagnetism based on measured values of the multiple magnetic sensors for geomagnetic field cancellation 2, determines a current for the active shield coil 9 so as to generate a magnetic field for canceling the fluctuating magnetic field based on measured values of the multiple magnetic sensors for active shield 3, and outputs a control signal corresponding to each of the determined currents; and the coil power supply 6 that outputs a current to each of the geomagnetic field nulling coil and the active shield coil 9 in response to the control signal output from the control device 5.

In the magnetoencephalographs M1 and M2 according to the present embodiment, the magnetic field relevant to the geomagnetism and the fluctuating magnetic field at the position of each of the multiple optically pumped magnetometers 1A for measuring the brain's magnetic field are measured. Then, in the magnetoencephalographs M1 and M2, the current for the geomagnetic field nulling coil is determined so as to generate a magnetic field for canceling the magnetic field relevant to the geomagnetism based on the multiple measured values of the magnetic field relevant to the geomagnetism, the current for the active shield coil 9 is determined so as to generate a magnetic field for canceling the fluctuating magnetic field based on the multiple measured values of the fluctuating magnetic field, and the control signal corresponding to each of the determined currents is output. Then, when the current corresponding to the control signal is output to each of the geomagnetic field nulling coil and the active shield coil 9, a magnetic field is generated in each coil. At the positions of the multiple optically pumped magnetometers 1A, the magnetic field relevant to the geomagnetism is canceled by the magnetic field generated in the geomagnetic field nulling coil, and the fluctuating magnetic field is canceled by the magnetic field generated in the active shield coil 9. Therefore, since the magnetic field relevant to the geomagnetism and the fluctuating magnetic field at the positions of the multiple optically pumped magnetometers 1A are canceled, the multiple optically pumped magnetometers 1A can measure the brain's magnetic field in a state in which the influence of the magnetic field relevant to the geomagnetism and the influence of the fluctuating magnetic field are avoided. According to such magnetoencephalographs M1 and M2, the brain's magnetic field can be measured with high accuracy without using the magnetic shield room.

The geomagnetic field nulling coil may include the geomagnetism nulling coil 7 for canceling a magnetic field of the geomagnetism and the gradient magnetic field nulling coil 8 for canceling a gradient magnetic field of the geomagnetism. The control device 5 may determine a current for the geomagnetism nulling coil 7 so that an average value of the measured values of the multiple magnetic sensors for geomagnetic field cancellation 2 approaches zero and determine a current for the gradient magnetic field nulling coil 8 so that a deviation from the average value of the measured values of the multiple magnetic sensors for geomagnetic field cancellation 2 is minimized. In such a configuration, uniform magnetic field cancellation (0th-order cancellation) is performed by controlling the current for the geomagnetism nulling coil 7, and gradient magnetic field cancellation (first-order cancellation) considering the difference between the positions of the optically pumped magnetometers 1A is performed by controlling the current for the gradient magnetic field nulling coil 8. In this manner, since the geomagnetism and the gradient magnetic field of the geomagnetism are canceled stepwise, the magnetic field relevant to the geomagnetism can be canceled with high accuracy.

Each of the geomagnetism nulling coil 7 and the gradient magnetic field nulling coil 8 may be a pair of coils arranged with the multiple optically pumped magnetometers 1A interposed therebetween.

According to such a configuration, the magnetic field relevant to the geomagnetism at the positions of the multiple optically pumped magnetometers 1A interposed between a pair of geomagnetism nulling coils 7 and between a pair of gradient magnetic field nulling coils 8 is effectively canceled. In this manner, the magnetic field relevant to the geomagnetism can be appropriately canceled by a simple configuration.

The geomagnetic field nulling coil may include the coil systems 15, which are arranged so as to be perpendicular to each other and surround each of the multiple optically pumped magnetometers 1A, and the control device 5 may determine currents for the coil systems 15 for each of the multiple optically pumped magnetometers 1A so that the measured values of the multiple magnetic sensors for geomagnetic field cancellation 2 approach zero. According to such a configuration, the coil systems 15 are arranged for each of the multiple optically pumped magnetometers 1A so as to correspond to the components of the static magnetic field in the three directions (x axis, y axis, and z axis). Then, by controlling the current for each of the coil systems 15, a magnetic field that cancels each of the x-axis direction component, the y-axis direction component, and the z-axis direction component of the magnetic field relevant to the geomagnetism is generated for each of the multiple optically pumped magnetometers 1A, and the magnetic field relevant to the geomagnetism is canceled in the three directions. Therefore, since the current can be finely controlled for each of the multiple optically pumped magnetometers 1A, the cancellation accuracy of the magnetic field relevant to the geomagnetism is improved. In addition, since only the magnetic field relevant to the geomagnetism in a region relevant to the operation of the multiple optically pumped magnetometers 1A is canceled, it is possible to suppress an increase in power consumption due to unnecessary cancellation.

The control device 5 may determine a current for the active shield coil 9 so that an average value of the measured values of the multiple magnetic sensors for active shield 3 approaches zero. According to such a configuration, the fluctuating magnetic field at the positions of the multiple optically pumped magnetometers 1A is effectively canceled by controlling the current for the active shield coil 9. In this manner, the fluctuating magnetic field can be appropriately canceled by a simple configuration.

The multiple optically pumped magnetometers 1A may be axial gradiometers having a measurement region and a reference region in a direction perpendicular to a scalp and coaxially. According to such a configuration, since the influence of common mode noise is shown in each of the output result of the measurement region and the output result of the reference region, the common mode noise can be removed by acquiring the difference between the output results of both. Therefore, the measurement accuracy of the brain's magnetic field is improved.

The multiple optically pumped magnetometers 1A, the multiple magnetic sensors for geomagnetic field cancellation 2, and the multiple magnetic sensors for active shield 3 may be fixed to the helmet-type non-magnetic frame 4 attached to the head of a subject. According to such a configuration, the non-magnetic frame 4 attached to the head and each sensor fixed to the non-magnetic frame 4 move according to the movement of the head of the subject. Therefore, even when the head of the subject moves, it is possible to appropriately cancel the magnetic field relevant to the geomagnetism and the fluctuating magnetic field at the positions of the multiple optically pumped magnetometers 1A and measure the brain's magnetic field.

The electromagnetic shield 14 for shielding high-frequency electromagnetic noise may be further provided. According to such a configuration, it is possible to prevent high-frequency electromagnetic noise, which cannot be measured by the magnetoencephalograph, from entering the multiple optically pumped magnetometers 1A. As a result, the multiple optically pumped magnetometers 1A can be stably operated.

A brain's magnetic field measurement method according to the present embodiment includes: measuring a magnetic field relevant to geomagnetism at a position of each of multiple optically pumped magnetometers 1A; determining a current for a geomagnetic field nulling coil so as to generate a magnetic field for canceling the magnetic field relevant to the geomagnetism based on multiple measured values of the magnetic field relevant to the geomagnetism and outputting a control signal for geomagnetic field cancellation corresponding to the determined current; outputting a current to the geomagnetic field nulling coil in response to the control signal for geomagnetic field cancellation; measuring a fluctuating magnetic field at the position of each of the multiple optically pumped magnetometers 1A; determining a current for an active shield coil 9 so as to generate a magnetic field for canceling the fluctuating magnetic field based on multiple measured values of the fluctuating magnetic field and outputting a control signal for fluctuating magnetic field cancellation corresponding to the determined current;

outputting a current to the active shield coil 9 in response to the control signal for fluctuating magnetic field cancellation; and measuring a brain's magnetic field with the multiple optically pumped magnetometers 1A.

In the brain's magnetic field measurement method according to the present embodiment, the magnetic field relevant to the geomagnetism and the fluctuating magnetic field at the position of each of the multiple optically pumped magnetometers 1A for measuring the brain's magnetic field are measured. Then, in the brain's magnetic field measurement method, the current for the geomagnetic field nulling coil is determined so as to generate a magnetic field for canceling the magnetic field relevant to the geomagnetism based on the multiple measured values of the magnetic field relevant to the geomagnetism, and the control signal corresponding to the determined current is output. Then, when the current corresponding to the control signal is output to the geomagnetic field nulling coil, a magnetic field is generated in the geomagnetic field nulling coil. At the positions of the multiple optically pumped magnetometers 1A, the magnetic field relevant to the geomagnetism is canceled by the magnetic field generated in the geomagnetic field nulling coil. In addition, the current for the active shield coil 9 is determined so as to generate a magnetic field for canceling the fluctuating magnetic field based on the multiple measured values of the fluctuating magnetic field, and the control signal corresponding to the determined current is output. Then, when the current corresponding to the control signal is output to the active shield coil 9, a magnetic field is generated in the active shield coil 9. At the positions of the multiple optically pumped magnetometers 1A, the fluctuating magnetic field is canceled by the magnetic field generated in the active shield coil 9. As a result, since the magnetic field relevant to the geomagnetism and the fluctuating magnetic field at the positions of the multiple optically pumped magnetometers 1A are canceled, the multiple optically pumped magnetometers 1A can measure the brain's magnetic field in a state in which the influence of the magnetic field relevant to the geomagnetism and the influence of the fluctuating magnetic field are avoided. According to such a brain's magnetic field measurement method, the brain's magnetic field can be measured with high accuracy without using the magnetic shield room.

Determining the current for the geomagnetic field nulling coil so as to generate a magnetic field for canceling the magnetic field relevant to the geomagnetism may include: determining a current for the geomagnetism nulling coil 7 forming the geomagnetic field nulling coil so that an average value of the multiple measured values of the magnetic field relevant to the geomagnetism approaches zero; and determining a current for the gradient magnetic field nulling coil 8 forming the geomagnetic field nulling coil so that a deviation from the average value of the multiple measured values of the magnetic field relevant to the geomagnetism is minimized. In such a method, uniform magnetic field cancellation (0th-order cancellation) is performed by controlling the current for the geomagnetism nulling coil 7, and gradient magnetic field cancellation (first-order cancellation) considering the difference between the positions of the optically pumped magnetometers 1A is performed by controlling the current for the gradient magnetic field nulling coil 8. In this manner, since the geomagnetism and the gradient magnetic field of the geomagnetism are canceled stepwise, the magnetic field relevant to the geomagnetism can be canceled with high accuracy.

Determining the current for the geomagnetic field nulling coil so as to generate a magnetic field for canceling the magnetic field relevant to the geomagnetism may include determining currents for the coil systems 15, which are arranged so as to be perpendicular to each other and surround each of the multiple optically pumped magnetometers 1A, so that the multiple measured values of the magnetic field relevant to the geomagnetism approach zero. According to such a method, the coil systems 15 are arranged for each of the multiple optically pumped magnetometers 1A so as to correspond to the components of the static magnetic field in the three directions (x axis, y axis, and z axis). Then, by controlling the current for each of the coil systems 15, a magnetic field that cancels each of the x-axis direction component, the y-axis direction component, and the z-axis direction component of the magnetic field relevant to the geomagnetism is generated for each of the multiple optically pumped magnetometers 1A, and the magnetic field relevant to the geomagnetism is canceled in the three directions. Therefore, since the current can be finely controlled for each of the multiple optically pumped magnetometers 1A, the cancellation accuracy of the magnetic field relevant to the geomagnetism is improved. In addition, since only the magnetic field relevant to the geomagnetism in a region relevant to the operation of the multiple optically pumped magnetometers 1A is canceled, it is possible to suppress an increase in power consumption due to unnecessary cancellation.

### Modification Examples

The above description has been made in detail based on the embodiment of the present disclosure. However, the present disclosure is not limited to the embodiment described above. The present disclosure can be modified in various ways without departing from its gist.

Although the active shield coil 9 has been described as having a pair of active shield coils 9A and 9B, the active shield coil 9 may be arranged as a coil system for each OPM module 1 (optically pumped magnetometer 1A) like the coil system 15. In this case, the control device 5 determines a current for the active shield coil 9 so that a magnetic field opposite to the components of the fluctuating magnetic field in the three directions (x axis, y axis, and z axis) at the position of the optically pumped magnetometer 1A and having approximately the same magnitude as the components of the fluctuating magnetic field is generated. The control device 5 outputs a control signal corresponding to the determined current relevant to each of the active shield coils 9, which are arranged as a coil system, to the coil power supply 6.

What is claimed is:

1. A magnetoencephalograph, comprising:
multiple optically pumped magnetometers configured to measure a brain's magnetic field;
multiple magnetic sensors for geomagnetic field cancellation configured to measure a magnetic field relevant to geomagnetism at a position of each of the multiple optically pumped magnetometers;
multiple magnetic sensors for active shield configured to measure a fluctuating magnetic field at the position of each of the multiple optically pumped magnetometers;
a geomagnetic field nulling coil for cancelling the magnetic field relevant to the geomagnetism;
an active shield coil for cancelling the fluctuating magnetic field;
a control device configured to determine a current for the geomagnetic field nulling coil so that the geomagnetic field nulling coil generates a magnetic field for canceling the magnetic field relevant to the geomagnetism based on measured values of the multiple magnetic sensors for geomagnetic field cancellation, determine a current for the active shield coil so that the active shield coil generates a magnetic field for canceling the fluctuating magnetic field based on measured values of the multiple magnetic sensors for active shield, and output a control signal corresponding to each of the determined currents; and
a coil power supply configured to output a current to each of the geomagnetic field nulling coil and the active shield coil in response to the control signal output from the control device.

2. The magnetoencephalograph according to claim 1,
wherein the geomagnetic field nulling coil includes a geomagnetism nulling coil for cancelling a magnetic field of the geomagnetism and a gradient magnetic field nulling coil for cancelling a gradient magnetic field of the geomagnetism, and
the control device determines a current for the geomagnetism nulling coil so that an average value of the measured values of the multiple magnetic sensors for geomagnetic field cancellation approaches zero, and determines a current for the gradient magnetic field nulling coil so that a deviation from the average value of the measured values of the multiple magnetic sensors for geomagnetic field cancellation is minimized.

3. The magnetoencephalograph according to claim 2,
wherein each of the geomagnetism nulling coil and the gradient magnetic field nulling coil is a pair of coils arranged with the multiple optically pumped magnetometers interposed therebetween.

4. The magnetoencephalograph according to claim 1,
wherein the geomagnetic field nulling coil includes coil systems arranged to be perpendicular to each other and to surround each of the multiple optically pumped magnetometers and configured to apply magnetic fields in three directions perpendicular to each other, for each of the multiple optically pumped magnetometers, and
the control device determines currents for the coil systems for each of the multiple optically pumped magnetometers so that the measured values of the multiple magnetic sensors for geomagnetic field cancellation approaches zero.

5. The magnetoencephalograph according to claim 1,
wherein the control device determines a current for the active shield coil so that an average value of the measured values of the multiple magnetic sensors for active shield approaches zero.

6. The magnetoencephalograph according to claim 1,
wherein the multiple optically pumped magnetometers are axial gradiometers having a measurement region and a reference region in a direction perpendicular to a scalp and coaxially.

7. The magnetoencephalograph according to claim 1,
wherein the multiple optically pumped magnetometers, the multiple magnetic sensors for geomagnetic field cancellation, and the multiple magnetic sensors for active shield are fixed to a non-magnetic frame of helmet-type attached to a head of a subject and having a relative permeability close to 1 so that a magnetic field distribution is not affected.

8. The magnetoencephalograph according to claim 1, further comprising:
an electromagnetic shield for shielding high-frequency electromagnetic noise.

9. A brain's magnetic field measurement method, comprising:
measuring a magnetic field relevant to geomagnetism at a position of each of multiple optically pumped magnetometers;
determining a current for a geomagnetic field nulling coil so that the geomagnetic field nulling coil generates a magnetic field for canceling the magnetic field relevant to the geomagnetism based on multiple measured values of the magnetic field relevant to the geomagnetism and outputting a control signal for geomagnetic field cancellation corresponding to the determined current;
outputting a current to the geomagnetic field nulling coil in response to the control signal for geomagnetic field cancellation;
measuring a fluctuating magnetic field at the position of each of the multiple optically pumped magnetometers;
determining a current for an active shield coil so that the active shield coil generates a magnetic field for canceling the fluctuating magnetic field based on multiple measured values of the fluctuating magnetic field and, outputting a control signal for fluctuating magnetic field cancellation corresponding to the determined current;
outputting a current to the active shield coil in response to the control signal for fluctuating magnetic field cancellation; and
measuring a brain's magnetic field with the multiple optically pumped magnetometers.

10. The brain's magnetic field measurement method according to claim 9,
wherein determining the current for the geomagnetic field nulling coil so that the geomagnetic field nulling coil generates a magnetic field for canceling the magnetic field relevant to the geomagnetism includes:
determining a current for a geomagnetism nulling coil forming the geomagnetic field nulling coil so that an average value of the multiple measured values of the magnetic field relevant to the geomagnetism approaches zero; and
determining a current for a gradient magnetic field nulling coil forming the geomagnetic field nulling coil so that a deviation from the average value of the multiple measured values of the magnetic field relevant to the geomagnetism is minimized.

11. The brain's magnetic field measurement method according to claim 9,
wherein determining the current for the geomagnetic field nulling coil so that the geomagnetic field nulling coil generates a magnetic field for canceling the magnetic field relevant to the geomagnetism includes:
determining currents for coil systems arranged to be perpendicular to each other and to surround each of the multiple optically pumped magnetometers, so that the multiple measured values of the magnetic field relevant to the geomagnetism approach zero.
